(19) United States
(12) Patent Application Publication
(10) Pub. No.: US 2012/0011620 A1
(43) Pub. Date: Jan. 12, 2012

(54) Algae Strain for Biodiesel Fuel Production

(75) Inventors: Andrew S. Gordon, Norfolk, VA (US); Patrick G. Hatcher, Suffolk, VA (US)

(73) Assignee: Old Dominion University, Norfolk, VA (US)

(21) Appl. No.: 13/195,182

(22) Filed: Aug. 1, 2011

Related U.S. Application Data
(63) Continuation-in-part of application No. 12/336,707, filed on Dec. 17, 2008.
(60) Provisional application No. 61/015,926, filed on Dec. 21, 2007.

(51) Int. Cl. A01H 13/00

(52) U.S. Cl. 800/296

(57) ABSTRACT

The present invention is a Desmodesmus strain of use in biodiesel production.

### INTRODUCTION

This application is a continuation-in-part of U.S. patent application Ser. No. 12/336,707, filed Dec. 17, 2008, which claims priority to U.S. Provisional Application No. 61/015,926, filed Dec. 21, 2007, which are incorporated herein by reference.

### BACKGROUND OF THE INVENTION

The recent emphasis on finding alternative energy sources to fuel the energy needs of the United States and the world is leading to an accelerated search for new fuels or new sources of fuel. Producing a liquid fuel from biomass, or biofuel, is an important focus of many alternative energy strategies. Ethanol production from plant biomass is one example of this. Another example of a newer fuel is biodiesel. Refined vegetable oils have been the typical starting materials for the production of biodiesel. Biodiesel can be produced from the oils of many plants. Biodiesel is an alternative, non-toxic, biodegradable and renewable diesel fuel. These characteristics of biodiesel reduce the emission of carbon monoxide, hydrocarbons, and particulate matter in the exhaust gas compared to diesel fuel.

Biodiesel is commonly referred to as fatty acid methyl esters (FAMEs), which are usually obtained from oils extracted from soybean, sunflower, rapeseed or even waste cooking oil. Biodiesel production relies on a chemical reaction called transesterification that transforms esters such as triglycerides into mono alkyl esters. Conventionally, this reaction requires a large excess of methanol, or in some cases ethanol, and an acid or a base catalyst under heated conditions.

### SUMMARY OF THE INVENTION

The present invention features an algal strain of the genus Desmodesmus, which was selected for growing under high nutrient conditions. The algal strain is characterized as having a fatty acid methyl ester content of 2.6% as determined by nuclear magnetic resonance analysis, a nitrogen content of 11.3% and a carbon content of 46.3%. In one embodiment, the Desmodesmus strain has an algaenan content of 5-10%. In another embodiment, the Desmodesmus strain has a ribosomal RNA sequence comprising SEQ ID NO:1.

### DETAILED DESCRIPTION OF THE INVENTION

Microalgae are capable of producing about thirty times the amount of oil per unit area of land, compared to terrestrial crops. The per unit area yield of oil from algae is estimated to be from between 5,000 to 20,000 gallons per acre, per year (4.6 to 18.4 l/m² per-year); this is 7 to 30 times greater than the next best crop, Chinese tallow (699 gallons). Due to the high growth efficiency of microalgae, the microalgae can efficiently recycle the inorganic carbon released from the petroleum combustion. For these reasons, algae are an ideal source from which to produce biodiesel.

Disclosed herein is an algal strain from the genus Desmodesmus. The algal strain of this invention was selected for its ability to grow under high nutrient conditions and maintain a stable population in a raceway pond for an extended period of time. Based upon the analysis described herein the instant strain is characterized as having a fatty acid methyl ester content of 2.6% as determined by nuclear magnetic resonance analysis, a nitrogen content of 11.3%, a carbon content of 46.3%, and an algaenan content of 5-10%. Given the growth and elemental composition of this strain, the instant algal strain is of particular use as a biomass source for biofuel lipids and/or biodiesel fuel production.

The instant strain can be grown in an aqueous solution that includes dissolved macronutrients and micronutrients. Macronutrients include nitrogen, phosphorus, and potassium, which are typically consumed in relatively large quantities by algae. In addition, the growth medium may provide secondary nutrients such as calcium, sulfur, and/or magnesium. Naturally occurring fertilizers may be dissolved into the growth medium as a source of macronutrients. Non-limiting examples of naturally occurring fertilizers include manure, slurry, worm castings, peat, seaweed, sewage, mine rock phosphate, sulfate of potash, limestone, and guano. Non-limiting examples of other compounds containing nutrients that may be dissolved into the growth medium include ammonia, ammonium, nitrate, urea, phosphate, and combinations thereof. Other non-limiting examples of macronutrient-containing compounds suitable for use in the growth medium include ammonium chloride, ammonium sulfate, mono-ammonium phosphate, diammonium phosphate, ammonium nitrate, sodium nitrate, potassium nitrate, calcium phosphate, super phosphate, triple super phosphate, and potassium chloride.

Micronutrients are defined herein as nutrients essential to the growth of an algal colony that may be included in the growth medium in relatively small or trace quantities. Non-limiting examples of suitable micronutrients for the growth medium include elements such as calcium, magnesium, sulfur, iron, copper, manganese, barium, zinc, chlorine, vanadium, selenium, sodium, molybdenum or any other element that may be beneficial to the growth of the algal colony.

The growth medium may further include other compounds depending on the nutritional needs of the algal colony. For example, methionine may be included in the growth medium to enhance the growth, lipid content and/or protein content of the instant algal strain (see, US 2011/0020914). Other non-limiting examples of compounds that may be included in the growth medium include vitamins, fungicides, bactericides, herbicides, and insecticides.

The instant microalgal strain can be propagated using various systems. For example, an open pond system or a photobioreactor can be used to provide light and carbon dioxide to the growing algal colony. An open pond system, as defined herein, includes a container that is typically relatively shallow in depth that further includes an exposed upper surface that is in direct contact with the surrounding atmosphere. The growth medium may be introduced to the open pond by methods including draining the pond and replacing with growth medium, or adding the compounds included in the growth medium to the existing water in the pond in amounts suitable transform the composition of the pond water into the desired growth medium composition. The microalgae may be introduced into the open pond system at a relatively low concentration and allowed to grow over a period of time sufficient to yield the desired cell density.

The open pond system may be completely exposed to the surrounding atmosphere. Alternatively, the open pond system may be partially enclosed using materials that are capable of transmitting adequate amounts of sunlight and fresh air to the exposed surface of the growth medium. For example, the open pond system may be sheltered by a transparent roof to prevent contamination of the growth medium by contaminants such as rainwater, pollen, fungal spores, and insects.

The open pond system may be a stationary body of water, or the open pond system may incorporate stirring devices such as paddle wheels or circulation pumps to mix the culture medium. In one embodiment, the open pond system may be a raceway pond in which the growth medium is directed by paddle wheels or circulation pumps through a continuous aquatic circuit containing a series of interconnected ponds. In particular embodiments, the instant algal strain is grown in a raceway containing one or more precipitators positioned along the raceway. See, e.g., US 2010/0031561.

A photobioreactor, as defined herein, is a closed system containing the algal strain. A photobioreactor typically includes a translucent container in which the algal strain is placed, along with a light source. The algae within the photobioreactor use the light from the light source in photosynthetic processes to actively grow and divide. Carbon dioxide for photosynthetic processes may be supplied to the algae passively by dissolving carbon dioxide gas into the growth medium via an exposed surface of the growth medium, or carbon dioxide may be actively supplied to the algae. For example, carbon dioxide gas may be bubbled through the growth medium by a gas line connected to a carbon dioxide gas source. Alternatively, carbon dioxide may be supplied to the growth medium by the introduction of chemical reagents including acids such as hydrochloric acid and metal carbonates such as calcium carbonate that produce carbon dioxide via chemical reactions

Photobioreactors may be run in a batch mode, in which the algae are introduced into a container, and grown in the same container until harvest. The container may also incorporate stirring or mixing in order to enhance the uptake of nutrients to the algae. The container may be stirred continuously or periodically.

Alternatively, photobioreactors may operate in a continuous mode, in which fresh growth medium is introduced to the container of the photobioreactor continuously, and algae are continuously harvested. The rate of addition of the fresh growth medium and the rate of harvest of algae are approximately matched to prevent significant depletion or accrual of algae and growth medium within the photobioreactor.

The photobioreactor may additionally control other growth conditions such as the temperature and pH of the growth medium, and limit the presence of other algal species and/or other organisms.

Algal growth can be expressed as any reasonable measure of cell density known in the art including the number of algal cells per unit volume of growth medium, the wet weight of the algal cells per unit volume of growth medium, and the dry weight of the algal cells per unit volume. In an exemplary embodiment, algal growth can be determined by estimating the density of the algal cells in the growth medium using devices known in the art including, but not limited to, a haemocytometer, a microscope with a counting chamber, a spectrophotometer, a fluorometer, and a colorimeter. In another embodiment, algal growth can also be expressed as a growth rate, defined herein as the change in growth per unit time.

In yet another aspect, the growth of the instant algal strain can be expressed as a characteristic of the individual algal cells, including but not limited to the average lipid content of the algal cells, the average protein content of the algal cells, and combinations thereof. The average lipid content of the algal cells may be estimated as described herein or using any other known techniques including but not limited to fluorometric measurements of algal cells in which the lipids have been dyed with a lipid-indicating dye such as Nile Red (9-diethylamino-5H-benzophenoxazine-5-one). The average protein content of the algal cells may be estimated using devices and methods known in the art including but not limited to a spectrophotometer to measure the absorbance of light at a selected wavelength of algal cells in which the protein is dyed with a protein-indicating dye such as COOMASSIE Blue G dye.

As indicated, the instant algal strain is of use in biodiesel fuel production. As used herein, “biodiesel fuel” refers to any fuel, fuel additive, aromatic and aliphatic compound derived from a biomass disclosed herein. When used as a biomass source for biodiesel fuel production, the instant strain can be grown as described above, and collected or harvested, e.g., by high voltage pulse-assisted aggregation (see, US 2011/0003350). The harvested material can then be used directly as reactor feedstock or processed further by well-known methods to convert it into reactor feedstock. For example, algae can be used directly, partially dried, completely dried, or dried and partially reconstituted in water. However, it has been recognized that drying increases the yield of fatty acid alkyl esters (FAAEs), the essential biodiesel component. Even though dried algae may be an ideal choice to feed a reactor considering the ease of use and probable higher biodiesel production, the drying procedure, takes time. The drying procedure may also require energy if freeze-drying is used. Lipids can also be degraded if the algal matter is left exposed to air too long.

The dried or partially dried biomass is subsequently fed to a reactor by means well-known in the art. The biomass may be conveyed, augered or sprayed, for example. The reactor may be of any type known in the art that can operate at the temperatures required. The configuration of the reactor is not consequential and any reaction chamber can be used.

As one embodiment of a process for biodiesel production, transesterification occurs. Transesterification is the process of exchanging the alkoxy group of an ester compound with another alkoxy group. The biomass contains glycerides that undergo hydrolysis in the reactor during transesterification. The glycerides may be mono-, di- or triglycerides. The ester links are severed during hydrolysis, producing free fatty acids. The transesterification process continues with the alkylation of the freed fatty acids. Methylation in particular refers to the alkylation process used to describe the delivery of a CH₃ group. A non-limiting example of an alkylation reagent is tetramethylammonium hydroxide (TMAH). Other non-limiting suitable alkylation reagents include tetrabutylammonium hydroxide, trimethylphenylammonium hydroxide, tetramethylammonium hydroxide, (m-trifluoromethylphenyl)trimethylammonium hydroxide, mixtures thereof and the like.

TMAH is a quaternary ammonium salt that can transesterify the biomass in one step. It can hydrolyze triglycerides and methylate the fatty acids simultaneously at the proper temperature, thus directly producing fatty acid methyl esters, or FAMEs. The by-products may include glycerol, water, trimethylamine, methanol or other water soluble compounds that can be easily separated by density or volatility. TMAH thermally decomposes to trimethyl amine plus methanol in the following equation:

(CH₃)₄NOH->(CH₃)₃N+CH₃OH

The trimethyl amine (TMA) by-product to which the TMAH is converted may be recycled and converted back to TMAH. As an example, the TMA can be reacted with methyl chloride gas in water to produce tetramethylammonium chloride (TMAC) as disclosed in U.S. Pat. No. 4,845,289. Methanol reacts with hydrochloric gas to produce methyl chloride and the methyl chloride reacts with TMA to produce TMAC. The TMAC can be passed through an anion exchange resin (OH form) to convert the TMAC to TMAH. Other byproducts may also be recovered and recycled or used in downstream processes. For example, the glyceryl backbone of the glycerides can be methylated to produce triglyme, a commercially usable product. Other byproducts may also be recovered and recycled or used in downstream processes.

FAAE yields are affected by the amount of alkylation reagent added to the reactor. Methods for determining the content of one or more fatty acid alkyl esters in a mixture are well known in the art and otherwise set forth herein. See, for example, U.S. Pat. Nos. 5,525,126; 6,855,838; and 6,965,044 and US 2007/0048848 and US 2003/0158074. Accordingly, the yield of one or more fatty acid alkyl esters resulting from the processes disclosed herein can be readily determined, alone or in combination with one or more well-known methods, such as those described in the cited references.

The transesterification takes place in a substantially oxygen-free environment. As used herein, “substantially oxygen-free environment” means that the oxygen content of the gaseous environment of a reaction, such as the transesterification reaction in the processes disclosed herein, is reduced compared to the oxygen content of air. Thus, substantially oxygen-free environment contemplates any amount of such reduction, including reduction of the oxygen to non-detectable levels. In this regard, substantially oxygen-free environment also contemplates that there may be residual oxygen remaining in the system. To achieve the substantially oxygen-free environment, the reactor can be purged with an inert gas using well-known means to reduce oxygen. Oxygen may also be reduced by preheating the reactor to the operating temperatures, thereby burning off the oxygen in the system. It is contemplated that the reduction in the oxygen is positively correlated to the amount of desired fatty acid alkyl ester yield. Thus, maximum reduction in the oxygen content results in higher yields of fatty acid alkyl ester. The optimal amount of the reduction of oxygen is determinable by monitoring the fatty acid alkyd ester yield from the processes of the invention by the methods described herein. In other words, the desired yield can be compared under any substantially oxygen-free environment and compared to the yield of transesterification under air. In one embodiment, the oxygen content of the gaseous environment of the transesterification reaction is selected from less than: 20%, 19%, 18%, 17%, 16%, 15%, 14%, 13%, 12%, 11%, 10%, 9%, 8%, 7%, 6%, 5%, 4%, 3%, 2%, 1% and undetectable amounts of the total. In another embodiment, the oxygen content is selected from less than 5%, 4%, 3%, 2%, 1% and undetectable amounts of the total. In another embodiment, the oxygen content is selected from less than 2% of the total. In another embodiment, the oxygen content is essentially zero, meaning it is undetectable.

The transesterification takes place under ambient pressure conditions. This reduces the cost of the process and increases the simplicity of the process. However, it is contemplated that the pressure can be reduced to less than ambient, allowing for a further reduction in operating temperature. It is also contemplated that the pressure may be increased to allow for a more efficient control of reactor conditions and product collection. The optimal amount of the reduction in pressure and/or temperature is determinable by monitoring the fatty acid alkyl ester yield from the processes of the invention by the methods described herein.

The transesterification occurs at a temperature sufficient to hydrolyze one or more lipid glycerides in the biomass and alkylate one or more fatty acids in the reaction. It has been shown that the yields of FAMEs produced at temperatures of 250 and 350° C. are about the same, approximately 3.2%. The yield was the highest (4.43%) at 450° C., and the lowest at 550° C. The low yield at 550° C. indicates that some of the FAMEs might be degraded at the higher temperature. It should be noted that although 450° C. achieves the optimum biodiesel yield in this particular process embodiment and at atmospheric conditions, lower temperatures may be used to provide suitable yields under different conditions, such as at pressures below atmospheric. Further, economics and energy requirements may make a lower temperature more favorable depending on the associated product yield. It should be noted that product yield, measured by methods discussed herein, may be optimized by varying at least one of temperature, pressure, and oxygen level. Therefore, in one embodiment, it is contemplated that temperatures as low as 100° C. will produce the desired yield when at least one of pressure and oxygen level is adjusted. In another embodiment, the temperature is selected from the range of 100° C. to 550° C., 150° C. to 500° C., 200° C. to 450° C., 250° C. to 400° C., or 300° C. to 350° C.

A second embodiment of using the instant algal strain in a process of direct conversion to biodiesel fuel is described below. The second embodiment is similar to the first embodiment. Therefore, descriptions of like steps and elements will not be repeated.

The reactor feed stock of the second embodiment includes a glyceride-based oil and an alkylation reagent. The feed stock is mixed and loaded into a reactor. The reactor has temperature control to maintain the reactor at a desired temperature. Inert gas sweeps the reactor to maintain a substantially oxygen free environment. At the desired temperature, transesterification occurs. The glycerides of the feed stock oil are hydrolyzed and the fatty acids are alkylated, directly producing FAAEs.

The glyceride-based oil can be extracted from the algal biomasses by conventional means known to those skilled in the art. During transesterification, the glycerides of the oil undergo hydrolysis in the reactor during transesterification. The glycerides may be mono-, di- or triglycerides. The ester links are severed during hydrolysis, producing free fatty acids. The transesterification process continues with the alkylation of the freed fatty acids. One alkylation reagent that can be used in the embodiments disclosed herein is tetramethylammonium hydroxide (TMAH). However, it is to be understood that the alkylation reagent is not limited to TMAH and may be other suitable alkylation reagents, examples of which include tetrabutylammonium hydroxide, trimethylphenylammonium hydroxide, tetraethylammonium hydroxide, (m-trifluoro-methylphenyl)trimethylammonium hydroxide and the like.

TMAH hydrolyzes the glycerides and methylates the fatty acids simultaneously at the proper temperature, following the same reaction equation disclosed in reference to the first embodiment. The by-products may include glycerol, water, trimethyl amine, methanol or other water soluble compounds that can be easily separated by density or volatility. The trimethylamine by-product to which the TMAH is converted may be recycled and converted back to TMAH as described above. Other byproducts may also be recovered and recycled or used in downstream processes

The reaction of the second embodiment occurs in the substantially oxygen-free environment at ambient pressure and sufficient temperature, as discussed in reference to the first embodiment. The volatile FAMEs are recovered with the same means discussed above.

The invention is described in greater detail by the following non-limiting examples.

### Example 1

### Isolation and Characterization of Strain 4N2

To obtain an alga that could be successfully maintained in open cultivation ponds, algae were grown in a high nutrient medium in an open tank in a greenhouse. Observations of algal community dynamics and laboratory isolation of various algae from the tank yielded several algal strains, including one designated 4N2. Based on morphology, 4N2 was identified as a member of the Desmodesmus algal group. These algae grow well in high nutrient conditions and have lipid content in the 10-20% range. Analysis of the rRNA gene of 4N2 indicated that it was similar, though not identical, to Desmodesmus asymmetricus.

Studies that have been done with this isolate include analysis of lipid content (lab and raceway), algaenan content (raceway, 5-10%), rRNA sequence, and growth studies related to morphological variability. Specifically, universal ribosomal primers ITS4 and ITS5 were used to sequence an approximately 630 bp region including partial 18s, complete ITS1, complete 5.8s, complete ITS2 and partial 26s genes (Innis, et al. (1990) PCR Protocols. A Guide to Methods and Applications. San Diego: Academic. 482 pp.).

The sequence of this ribosomal sequence for algae strain 4N2 was:

(SEQ ID NO: 1)
TCCTTATTTTTTGTGGTACCGACGTTTTGGTCAACACACGCAAGTGTG
TGGCCTACTAACCTACACACCATTGACCAACCAATAATCAAACCAAAC
TCTGAAGCTTTGGCTGCTGTTAATCGGCAGTTTTAACAAAGAACAACT
CTCAACAACGGATATCTTGGCTCTCGCAACGATGAAGAACGCAGCGAA
ATGCGATACGTAGTGTGAATTGCAGAATTCCGTGAACCATCGAATCTT
TGAACGCATATTGCGCTCGACCCCTCGGGGAAGAGCATGTCTGCCTCA
GCGTCGGTTTACACCCTCACCCCACTTCCCTCACAGGAAGCGCTTGCT
GCGTCGTTTGACCAGCAACTGGGATGGATCTGGCCCTCCCAATCGAAG
CAATTCGATTGGGTTGGCTGAAGCACAGAGGCTTAAACTGGGACCCGT
ACCGGGCTCAACTGGATAGGTAGCAACACCCTCGGGTGCCTACACGAA
GTTGTGTCTGAGGACCTGGTTAGGAGCCAAGCAGGAAACGTGGAAACA
CGTACTCTGTATTCGACCTGAGCTCAGGCAAGGCTACCCGCTGAACTT
AAGCATATCAATAAAGCGGGAGGAA

The 4N2 strain was analyzed for carbon, nitrogen, and lipid content to determine whether it would be ideal for growth in raceways and conversion into biodiesel. For analysis of carbon, nitrogen, and lipid content, cells were grown in BG-11 medium at room temperature under artificial light (2 F40T12/DX 40 watt fluorescent bulbs). The results of this analysis indicated that 4N2 had a carbon content of 46.3% and a nitrogen content of 11.3%. Based upon gas chromatography-mass spectrometry analysis (GC-MS), 4N2 had a lipid content of 11.3% using a DCM/MeOH extraction method or 12.5% using a DCM/MeOH Soxhlet extraction method. Given that solvent extraction methods can overestimate lipid content by including pigment along with fatty acids, total lipid content was also determined by high resolution—magic angle spinning nuclear magnetic resonance (NMR) analysis. NMR analysis indicated that 4N2 had a fatty acid methyl ester (FAME) content of 2.6%.

Large volumes (25 gallons) of 4N2 were grown and transferred to an algae farm, where it was further propagated into approximately 30,500 gallon tanks. These tanks were the used to inoculate a raceway. About one year after inoculation of the raceway, a genetic analysis showed that D. asymmetricus was the dominant alga present. Subsequent microscopic analyses of the algal community in the raceway showed a seasonal succession of dominant algal populations, but Desmodesmus always maintained a significant presence and was generally the dominant population.

The original culture of 4N2 was maintained in the laboratory and given a generation time of one to three per day, depending on growth conditions, the stain has been maintained for approximately 1095 to 3285 generations. Given that this strain can be reliable propagated under laboratory conditions and grows well in the high nutrient conditions of a raceway, the 4N2 strain is of particular use in biodiesel production.

What is claimed is:

1. A Desmodesmus strain characterized as having a fatty acid methyl ester content of 2.6% as determined by nuclear magnetic resonance analysis, a nitrogen content of 11.3% and a carbon content of 46.3%.

2. The Desmodesmus strain of claim 1, further characterized as having an algaenan content of 5-10%.

3. The Desmodesmus strain of claim 1, further characterized as having a ribosomal RNA sequence comprising SEQ ID NO:1.


SEQUENCE LISTING (1 sequence)

SEQ ID NO: 1 - DNA, 601 bp, Desmodesmus sp.
tccttattttttgtggtaccgacgttttggtcaacacacgcaagtgtgtggcctactaacctacacaccattgaccaaccaataatcaaaccaaactctgaagctttggctgctgttaatcggcagttttaacaaagaacaactctcaacaacggatatcttggctctcgcaacgatgaagaacgcagcgaaatgcgatacgtagtgtgaattgcagaattccgtgaaccatcgaatctttgaacgcatattgcgctcgacccctcggggaagagcatgtctgcctcagcgtcggtttacaccctcaccccacttccctcacaggaagcgcttgctgcgtcgtttgaccagcaactgggatggatctggccctcccaatcgaagcaattcgattgggttggctgaagcacagaggcttaaactgggacccgtaccgggctcaactggataggtagcaacaccctcgggtgcctacacgaagttgtgtctgaggacctggttaggagccaagcaggaaacgtggaaacacgtactctgtattcgacctgagctcaggcaaggctacccgctgaacttaagcatatcaataaagcgggaggaa